(19) United States
(12) Patent Application Publication
(10) Pub. No.: US 2009/0042940 A1
(43) Pub. Date: Feb. 12, 2009

(54) METHODS AND COMPOSITIONS USING CHOLINESTERASE INHIBITORS

(75) Inventors: John Ieni, Bloomfield, NJ (US); Raymond Pratt, Baltimore, MD (US)

Correspondence Address: VENABLE LLP, P.O. BOX 34385, WASHINGTON, DC, 20043-9998, US

(73) Assignee: Eisai Co., Ltd., Tokyo (JP)

(21) Appl. No.: 12/192,856

(22) Filed: Aug. 15, 2008

Related U.S. Application Data
(62) Division of application No. 10/988,600, filed on Nov. 16, 2004, now abandoned.
Application No. 10/988,600 is a continuation of application No. PCT/US03/15279, filed on May 16, 2003.
(60) Provisional application No. 60/447,724, filed on Feb. 19, 2003.
(60) Provisional application No. 60/380,852, filed on May 17, 2002.

(51) Int. Cl. A61K 31/445

(52) U.S. Cl. 514/319

(57) ABSTRACT

The invention provides methods for treating and/or preventing Alzheimer's disease, psychiatric illnesses, encephalitis, meningitis, fetal alcohol syndrome, Karsakoff's syndrome, anoxic brain injury, cardiopulmonary resuscitation injuries, diabetes, Sjogren's syndrome, mental retardation, developmental delay, menopause, strokes, macular degeneration, neuronal loss associated with macular degeneration, sleep disorders, severe Alzheimer's disease, jet lag, post-traumatic stress disorder, anxiety disorders, panic attacks, obsessive-compulsive disorder, amnesia, and other disorders by administering to a patient in need thereof at least one cholinesterase inhibitor. The invention also provides novel pharmaceutical compositions that can be administered to the eyes or to the nose of patients. In one embodiment, the cholinesterase inhibitor is donepezil, a stereoisomer thereof and/or a pharmaceutically acceptable salt thereof. In other embodiments, the cholinesterase inhibitor can be one or more of phenserine, tolserine, phenethylnorcymserine, ganstigmine, epastigmine, tacrine, physostigmine, pyridostigmine, neostigmine, rivastigmine, galantamine, citicoline, velnacrine, huperzine, metrifonate, heptastigmine, edrophonium, TAK-147, T-82, and upreazine.

### RELATED APPLICATIONS

This application is a divisional of U.S. Non-Provisional Application No. 10/988,600 filed Nov. 16, 2004, which is a continuation of PCT Application No. PCT/US03/15279 filed May 16, 2003, which claims priority to U.S. Application No. 60/447,724 filed Feb. 19, 2003, and U.S. Application No. 60/380,852 filed May 17, 2002.

### FIELD OF THE INVENTION

The invention provides methods for treating and/or preventing cognitive impairments, dementia, and other disorders by administering a therapeutically effective amount of at least one cholinesterase inhibitor. A preferred cholinesterase inhibitor is donepezil, a stereoisomer thereof, and/or a pharmaceutically acceptable salt thereof.

### BACKGROUND OF THE INVENTION

Cholinesterase inhibitors are described in U.S. Pat. No. 4,895,841 and WO 98/39000, the disclosures of which are incorporated by reference herein in their entirety. The cholinesterase inhibitors described in U.S. Pat. No. 4,895,841 include donepezil hydrochloride or ARICEPT®, which has proven to be a highly successful drug for the treatment of Alzheimer's disease. There is a need in the art for new and improved treatments for other diseases, disorders, and syndromes that may be characterized by symptoms of cognitive impairments. The invention is directed to these, as well as other, important ends.

### SUMMARY OF THE INVENTION

The invention provides methods for treating and/or preventing Alzheimer's disease, vascular dementia, dementia associated with Parkinson's disease, visuospatial deficits, Williams syndrome, encephalitis, meningitis, fetal alcohol syndrome, Korsakoff's syndrome, anoxic brain injury, cardiopulmonary resuscitation injuries, diabetes, Sjogren's syndrome, mental retardation, developmental delay, menopause, pre-menstrual syndrome, strokes, macular degeneration, sleep disorders, cognitive impairments caused by high cholesterol levels, jet lag, post-traumatic stress disorder, anxiety disorders, panic attacks, obsessive-compulsive disorder, amnesia and other disorders by administering to a patient at least one cholinesterase inhibitor.

The invention provides novel nasally administrable pharmaceutical compositions comprising at least one cholinesterase inhibitor and a nasal delivery system.

The invention provides methods of treating migraines in a patient in need thereof by the nasal administration of a pharmaceutical composition comprising at least one cholinesterase inhibitor and a nasal delivery system.

The invention provides ophthalmic compositions comprising at least one cholinesterase inhibitor. The ophthalmic compositions of the invention can be used, for example, to treat macular degeneration, Sjogren's syndrome, and glaucoma.

The invention is described in more detail below.

### DETAILED DESCRIPTION OF THE INVENTION

“Patient” refers to animals, preferably mammals, more preferably humans. The term “patient” includes adults and children, and men and women. Children includes neonates, infants, and adolescents.

“Cognitive impairment” refers to an acquired deficit in one or more of memory function, problem solving, orientation and/or abstraction that impinges on a patient's ability to function independently.

“Dementia” refers to a global deterioration of intellectual functioning in clear consciousness, and is characterized by one or more symptoms of disorientation, impaired memory, impaired judgment, and/or impaired intellect. The symptoms of “dementia” are generally worse than, and can encompass, the symptoms of “cognitive impairment.”

The invention provides methods for treating and preventing cognitive impairments and/or dementia caused by radiation in a patient in need thereof by administering a therapeutically effective amount of at least one cholinesterase inhibitor. The radiation may be, for example, a radiation treatment used for cancer or an accidental exposure to radioactive materials.

The invention provides methods for treating and preventing visuospatial deficits in a patient in need thereof by administering a therapeutically effective amount of at least one cholinesterase inhibitor. Visuospatial deficits refer to problems with perceiving, processing and/or interpreting information through the visual system. Visuospatial deficits can include impairments in visuoperceptual abilities and visuoconstructive abilities.

The invention provides methods for treating and preventing Williams syndrome in a patient in need thereof by administering a therapeutically effective amount of at least one cholinesterase inhibitor. Williams syndrome is a developmental disorder that is characterized by visuospatial deficits. Most patients with Williams syndrome have mild or moderate mental retardation (mean IQ ranging from 55-60), but some have borderline normal intelligence or severe mental retardation.

The invention provides methods for treating and preventing cognitive impairments and/or dementia associated with or caused by encephalitis by administering to a patient in need thereof at least one cholinesterase inhibitor. “Encephalitis” refers to an inflammation of the brain. Symptoms can include a sudden fever, headache, vomiting, photophobia, stiff neck and back, confusion, drowsiness, clumsiness, unsteady gait, and irritability. Other symptoms include loss of consciousness, poor responsiveness, seizures, muscle weakness, sudden severe dementia, memory loss, withdrawal from social interaction, and impaired judgment.

The invention provides methods for treating and preventing cognitive impairments and/or dementia associated with or caused by meningitis by administering to a patient in need thereof a therapeutically effective amount of at least one cholinesterase inhibitor. “Meningitis” refers to an infection of the membranes that surround the brain and spinal cord. Symptoms can appear suddenly and can include a high fever, severe and persistent headache, stiff neck, nausea, and vomiting. Other symptoms can include confusion, sleepiness, and difficulty waking up. Symptoms of meningitis in infants can include irritability or tiredness, poor feeding and fever.

The invention provides methods for treating and preventing Sjogren's syndrome by administering to a patient in need thereof a therapeutically effective amount of at least one cholinesterase inhibitor. “Sjogren's syndrome” refers to a chronic autoimmune disorder in which immune cells attack and destroy the glands that produce tears and saliva. The symptoms of Sjogren's syndrome can include dry eyes and a dry mouth. Additional symptoms can include skin, nose, and/or vaginal dryness. Sjogren's syndrome can be associated with rheumatic disorders, such as rheumatoid arthritis, and can affect other organs of the body including the kidneys, blood vessels, lungs, liver, pancreas, and brain. In the methods of the invention, the cholinesterase inhibitors can be used systemically or by topical application to the eyes to treat Sjogren's syndrome.

The invention provides methods for treating and preventing cognitive impairments and/or dementia associated with or caused by fetal alcohol syndrome by administering to a patient in need thereof a therapeutically effective amount of at least one cholinesterase inhibitor. “Fetal alcohol syndrome” refers to the manifestation of specific growth, mental, and physical birth defects associated with a mother's high level of alcohol use during pregnancy. Symptoms of fetal alcohol syndrome can include slow intrauterine and neonatal growth with occasional diagnosis of failure to thrive, delayed development and evidence of mild to moderate mental retardation, facial abnormalities, skeletal abnormalities, tremor in the newborn infant, agitation and crying by the newborn infant, simian crease or other abnormal creases on the palm, growth deficiency, heart defects, vision difficulties, abnormal behavior such as short attention span, hyperactivity, poor impulse control, extreme nervousness and anxiety, and limb abnormalities.

The invention provides methods for treating and preventing cognitive impairments (e.g., memory impairment) associated with or caused by Korsakoff's syndrome by administering to a patient in need thereof at least one cholinesterase inhibitor. “Korsakoff's syndrome” is a syndrome in which the impairment of memory is out of proportion to other cognitive functions. Symptoms can include memory impairment (e.g., loss of memory, inability to form new memories), vision changes (such as double vision), loss of muscle coordination, symptoms that indicate alcohol withdrawal, and fabrication of stories.

The invention provides methods for treating and preventing cognitive impairments and/or dementia associated with or caused by anoxic brain injury by administering to a patient in need thereof at least one cholinesterase inhibitor. “Anoxic brain injury” refers to the injury to the brain caused by an absence or complete lack of oxygen supply to the brain's tissues. Symptoms can include seizures, muscle spasms or twitches, short-term memory loss, word-finding difficulties, visual disturbances, incoordination, inability to follow a sequence of commands, spasticity, weakness or paralysis, and neck stiffness. Severe symptoms include coma or unconsciousness for hours to days, weeks or months.

The invention provides methods for treating and preventing cognitive impairments and/or dementia caused by cardiopulmonary resuscitation by administering to a patient in need thereof a therapeutically effective amount of at least one cholinesterase inhibitor. “Cardiopulmonary resuscitation” refers to the administration of heart compression and artificial respiration to restore circulation and breathing. Possible post-resuscitation complications can include cognitive impairments, cardiovascular dysfunction, microcirculatory dysfunction, hypoxia and the release of toxic enzymes and free radicals, multiple organ dysfunction syndrome, seizures, systemic inflammatory response syndrome, septic shock, and sepsis syndrome.

The invention provides methods for treating and preventing cognitive impairments and/or dementia caused by or associated with diabetes by administering to a patient in need thereof at least one cholinesterase inhibitor. “Diabetes” refers to a disease of high blood sugar caused by too little insulin, resistance to insulin, or both. “Type 1 diabetes” occurs when the body makes little or no insulin and daily injections of insulin are required to live. “Type 2 diabetes” occurs when the pancreas does not make enough insulin to keep blood glucose levels normal. Symptoms of Type 1 diabetes can include increased thirst, increased urination, weight loss in spite of increased appetite, fatigue, nausea and vomiting. Symptoms of Type 2 diabetes can include increased thirst, increased urination, increased appetite, fatigue, blurred vision, slow healing infections, and sexual dysfunctions. Possible complications of diabetes can include heart disease, stroke, eye diseases (such as cataracts, glaucoma, or blindness), kidney disease leading to kidney failure, and nervous system disease. Cognitive impairments resulting from diabetes can include the loss of mental agility, poor concentration, disruptive behavior, fatigue, anxiety, tension, confusion, disorientation, aggression, memory problems, and mood changes.

The invention provides methods for treating and preventing mental retardation by administering to a patient in need thereof a therapeutically effective amount of at least one cholinesterase inhibitor. “Mental retardation” refers to the below-average general intellectual function with associated deficits in adaptive behavior that occurs before age 18. Symptoms of mental retardation can include failure to meet intellectual developmental markers, persistence of infantile behavior, lack of curiosity, decrease learning ability, and inability to meet educational demands of school.

The invention provides methods for treating and preventing developmental delay by administering to a patient in need thereof a therapeutically effective amount of at least one cholinesterase inhibitor. “Developmental delay” refers to a child who fails to achieve certain skills as quickly as expected, i.e., a child not reaching developmental bench marks at the usual age. Signs of developmental delay can include the delay in walking and other motor skills, the inability to walk, language delay or inability to learn, abnormalities of vision or hearing, behavioral problems, and seizures.

The invention provides methods for treating and preventing cognitive impairments (e.g., memory loss) caused by or associated with menopause by administering to a patient in need thereof at least one cholinesterase inhibitor. “Menopause” refers to the transition period in a woman's life when the ovaries stop producing eggs, menstrual activity decreases and eventually ceases, and the body decreases the production of the female hormones, estrogen and progesterone. The symptoms of menopause can include memory loss, hot flashes, skin flushing, mood changes, decreased libido, irregular menstrual periods, and vaginal dryness.

The invention provides methods for treating and preventing cognitive impairments (e.g., language development) and/or dementia caused by or associated with strokes by administering to a patient in need thereof at least one cholinesterase inhibitor. In another embodiment, the invention provides methods of treating post-stroke aphasia in a patient in need thereof by administering at least one cholinesterase inhibitor. A “stroke” refers to the loss of brain functions caused by a loss of blood circulation to areas of the brain. Symptoms of a stroke can include loss of movement (paralysis) of a body area, weakness, decreased sensation, numbness, decreased vision, language difficulties (aphasia) (such as slurred, thick or difficult speech, or the inability to speak), inability to understand speech and difficulty with reading or writing, inability to recognize or identify sensory stimuli, loss of memory, vertigo, loss of coordination, swallowing difficulties, personality changes, mood/emotion changes, consciousness changes, urinary incontinence, and cognitive decline such as dementia, impaired judgment and limited attention. Additional symptoms can include tongue problems, seizures, jerky movement, uncontrollable and/or dysfunctional movement, fainting, drooling, temporary absent breathing, and lack of sweating.

The invention provides methods for treating and preventing macular degeneration by administering to a patient in need thereof at least one cholinesterase inhibitor. In another embodiment, the invention provides methods for preventing the neuronal loss associated with macular degeneration by administering to a patient in need thereof at least one cholinesterase inhibitor. “Macular degeneration” refers to a disorder that affects the macula or the central part of the retina causing decreased visual acuity and possible loss of central vision. Symptoms of macular degeneration can include blurred, distorted, dim or absent central vision. In the methods of the invention, the cholinesterase inhibitors can be used systemically or by topical application of the eyes to treat macular degeneration or neuronal loss associated with macular degeneration.

The invention provides methods for treating and preventing sleep disorders by administering to a patient in need thereof at least one cholinesterase inhibitor. “Sleep disorders,” refer to a disruptive pattern of sleep that can include difficulty falling or staying asleep, falling asleep at inappropriate times, excessive total sleep time, or abnormal behaviors associated with sleep. Sleep disorders include, for example, REM (rapid eye movement) disorders, sleep onset with depression, age-related sleep disorders, narcolepsy, sleep deprivation, and REM-deprived sleep disorders. Symptoms of sleep disorders can include awakening in the night, difficulty falling asleep, excessive daytime drowsiness, loud snoring, episodes of stopped breathing, sleep attacks during the day, daytime fatigue, depressed mood, anxiety, difficulty concentrating, apathy, irritability, loss of memory or decreased memory, and lower leg movements during sleep. “REM sleep” refers to the occasional periods of active dreaming during sleep. “Age related sleep disorders” refer to the increased difficulty of falling asleep, the increase of awakenings, the less time spent in deep dreamless sleep, which can lead to confusion and other metal changes. “Narcolepsy” refers to a sleep disorder associated with uncontrollable sleepiness and frequent daytime sleeping.

In another embodiment, the invention provides methods for enhancing REM sleep by administering to a patient in need thereof at least one cholinesterase inhibitor. Enhancing REM sleep includes, for example, increasing the number of REM sleep episodes and/or increasing the duration of REM sleep episodes. Without intending to be bound by any theory of the invention, enhancing REM sleep may enhance memory consolidation and learning, and may improve a patient's mood.

In another embodiment, the invention provides methods for treating and/or preventing cognitive impairments and/or dementia associated with or caused by high cholesterol levels in a patient. Without intending to be bound by any theory of the invention, it is believed that high cholesterol levels cause cognitive impairments, such as impaired memory. High cholesterol generally refers to a cholesterol level greater than 200; about 215 or higher; about 225 or higher; or about 235 or higher.

The invention provides methods for treating and preventing jet lag by administering to a patient in need thereof at least one cholinesterase inhibitor. “Jet lag” refers to a physical reaction to a rapid change in time zones. Symptoms of jet lag can include disorientation, irritability, fatigue, swollen limbs and eyes, headaches, cold-like symptoms, and irregular bowels.

The invention provides methods for treating and preventing post-traumatic stress disorders by administering to a patient in need thereof at least one cholinesterase inhibitor. “Post-traumatic stress disorder” is a psychiatric illness that can occur following a traumatic event in which there is the threat of injury or death to the patient or someone else. Symptoms can include recurrent distressing memories of the event, recurrent dreams of the event, flashback episodes, bodily reactions to situations that remind the person of the traumatic event, the inability to remember important aspects of the trauma, lack of interest in normal activities, feelings of detachment, reduced expression of moods, irritability or outburst of anger, sleeping difficulties, difficulty concentrating, hypervigilance, paleness, heart palpitations, headache, fever, fainting, dizziness, and agitation. Some possible complications can include depression, anxiety, unusual phobia to things that are not usually frightening to other people, alcohol abuse and/or drug abuse.

The invention provides methods for treating and preventing anxiety disorders or panic attacks by administering to a patient in need thereof at least one cholinesterase inhibitor. “Panic attacks” refer to unexpected and repeated episodes of intense fear accompanied by physical symptoms that can include chest pain, heart palpitations, shortness of breath, dizziness or abdominal distress. Other symptoms can include terror, nausea, tingling or numbness in the hands, flushes or chills, sense of unreality, fear of losing control, going “crazy,” or doing something embarrassing, and fear of dying.

The invention provides methods for treating and preventing obsessive-compulsive disorder by administering to a patient in need thereof at least one cholinesterase inhibitor. “Obsessive-compulsive disorder” refers to an anxiety disorder characterized by the presence of obsessions or compulsions. An “obsession” refers to a recurrent or persistent thought that is intrusive or inappropriate. A “compulsion” is a repetitive behavior a patient feels driven to perform such as a physical action (i.e., handwashing) or a mental action (i.e., praying, repeating words, counting). Symptoms of the disorder include obsessions or compulsions that cause significant distress or interference with every day life, and are not due to medical illness or drug use.

The invention provides methods for treating and preventing cognitive impairments associated with patients who ingest or are exposed to MTPT by administering a therapeutically effective amount of at least one cholinesterase inhibitor. MTPT is a designer drug that can produce symptoms of Parkinson's disease in a patient who uses it or makes it.

The invention provides methods for treating and preventing amnesia by administering to a patient in need thereof at least one cholinesterase inhibitor. “Amnesia” refers to a disturbance in memory manifested by total or partial inability to recall past experiences. Symptoms of amnesia can include memory gaps, confusion, changes in emotion, difficulty in remembering recent events and/or events in the past, and disorientation. Cognitive impairments associated with amnesia can include difficulty thinking/concentrating, drops in IQ, and problems with fine/gross motor coordination.

The invention provides methods for treating Alzheimer's disease and/or delaying the onset of Alzheimer's disease in a patient in need thereof by administering a therapeutically effective amount of at least one cholinesterase inhibitor and at least one statin. In another embodiment, the invention provides methods for treating Alzheimer's disease and/or delaying the onset of Alzheimer's disease by administering a therapeutically effective amount of at least two cholinesterase inhibitors. In another embodiment, the invention provides methods for treating Alzheimer's disease and/or delaying the onset of Alzheimer's disease by administering a therapeutically effective amount of at least two cholinesterase inhibitors and at least one statin. The statin can be any in the art. Exemplary statins include fluvastatin, atorvastatin, simvastatin, pravastatin, lovastatin, cerivastatin, rosuvastatin, and the like. The cholinesterase inhibitor and statin can be administered separately or in the form of a composition.

The invention provides methods for treating and/or delaying the onset of vascular dementia (also known as cerebrovascular dementia) or dementia associated with Parkinson's disease in a patient in need thereof by administering a therapeutically effective amount of at least one cholinesterase inhibitor and at least one statin. In another embodiment, the invention provides methods for treating and/or delaying the onset of vascular dementia or dementia associated with Parkinson's disease by administering a therapeutically effective amount of at least two cholinesterase inhibitors. In another embodiment, the invention provides methods for treating and/or delaying the onset of vascular dementia or dementia associated with Parkinson's disease by administering a therapeutically effective amount of at least two cholinesterase inhibitors and at least one statin. The cholinesterase inhibitor and statin can be administered separately or in the form of a composition.

The invention provides methods for treating and/or delaying the onset of Alzheimer's disease, vascular dementia, Parkinson's disease, or cognitive impairments by administering to a patient in need thereof a therapeutically effective amount of at least one cholinesterase inhibitor and at least one anti-oxidant. The anti-oxidant can be any in the art. Exemplary anti-oxidants include vitamin E, BHA (i.e., butylated hydroxyanisole), BHT (i.e., butylated hydroxytoluene), vitamin C, budralazine, cadralazine, dihydralazine, endralazine, hydralazine, pildralazine, todralazine, glutathione, cysteine, N-acetyl-cysteine, P-carotene, ubiquinone, ubiquinol-10, tocopherols, coenzyme Q, superoxide dismutase, catalase, glutathione peroxidase, and the like. A preferred anti-oxidant is vitamin E. The anti-oxidants can be synthetic or natural. The cholinesterase inhibitor and the anti-oxidant can be administered separately or in the form of a composition.

The invention provides methods for treating and/or delaying the onset of Alzheimer's disease by administering a therapeutically effective amount of at least one cholinesterase inhibitor and an Alzheimer's vaccine. The Alzheimer's vaccine can be any in the art. In one embodiment, the Alzheimer's vaccine comprises an amyloid.

The invention provides methods for treating psychiatric disorders by administering to a patient in need thereof a therapeutically effective amount of at least one cholinesterase inhibitor. In another embodiment, the invention provides for methods treating psychiatric disorders by administering to a patient in need thereof a therapeutically effective amount of at least one cholinesterase inhibitor and at least one psychiatric medicine.

Any psychiatric medicine in the art can be used depending on the psychiatric illness that is being treated. Psychiatric medicines include, for example, antidepressant; anti-psychotic medications (e.g., pimozide, chlorpromazine, phenothiazines, butyrophenone, thioxanthines, haloperidol, sulpride, clozapine, sulpiride, tiapride, bifemelane, amisulpride, risperidone, olanzapine, quetiapine, polycarbophil, ziprasidone, aripiprazole, iloperidone, trifluoperazine, loxapine, molindone, fluphenazine, thiothixene, perphenazine, prochlorperizine, perphenazine/amitryptiline, mesoridazine, thioridazine); mood stabilizers (e.g., lithium, divalproex, gabapentin, carbamazepine, lamotrigine, topiramate); anti-anxiety medications (e.g., hydroxyzine, doxepin, venlafaxine, paroxetine, meprobamate, NGD 91-3, and benzodiazepines (such as alprazolam, flurazepam, oxazepam, triazolam, estazolam, chlordiazepoxide, lorazepam, quazepam, diazepam, tamazepam, clonazepam); stimulants (e.g., methylphenidate, dextroamphetamine, pemoline, dextroamphetamine/levoamphetamine), and the like.

Antidepressants include, for example, tricyclic antidepressants (e.g., amitriptyline, desipramine, imipramine, nortirptyline); serotonin-specific reuptake inhibitors (e.g., fluoxetine, paroxetine, sertraline, citalopram, fluvoxamine); monoamine oxidase inhibitors (e.g., phenelzine, tranylcypromine, isocarboxazid); other antidepressants (e.g., venlafaxine, nefazodone, bupropion, mirtazapine, trazodone, thioridazine, protriptyline), and the like. The psychiatric disorder can be any known in the art. Exemplary psychiatric disorders include obsessive-compulsive disorder, post-traumatic stress disorder, anxiety, panic attacks, schizophrenia, depression, mania, manic-depression (bipolar disorder), autism, dyslexia, apathy, delirium, attention deficit hyperactivity disorder, phobias, eating disorders (e.g., bulimia, anorexia), and the like.

The invention provide methods for treating and/or delaying the onset of Alzheimer's disease, vascular dementia, or dementia associated with Parkinson's disease by administering to a patient in need thereof at least one cholinesterase inhibitor and at least one NMDA receptor blocker. In another embodiment, the invention provides methods for treating and/or delaying the onset of Alzheimer's disease, vascular dementia, or dementia associated with Parkinson's' disease by administering to a patient in need thereof at least two cholinesterase inhibitors and at least one NMDA receptor blocker. Any NMDA receptor blocker in the art can be used. Exemplary NMDA receptor blockers include memantine, remacemide, dizocilipine, dextromethorphan, dextorphan, AP5, AP7 and the like. In one embodiment, the NMDA receptor blocker is memantine. The cholinesterase inhibitor and NMDA blocker can be administered separately or in the form of a composition.

The invention provide methods for treating memory loss, cognitive impairments or dementia; and for treating and/or delaying the onset of Alzheimer's disease, vascular dementia, or dementia associated with Parkinson's disease by administering to a patient in need thereof at least one cholinesterase inhibitor and at least one calcium channel blocker. In another embodiment, the invention provides methods for treating and/or delaying the onset of Alzheimer's disease, vascular dementia, or dementia associated with Parkinson's' disease by administering to a patient in need thereof at least two cholinesterase inhibitors and at least one calcium channel blocker. Any calcium channel blocker in the art can be used. Exemplary calcium channel blockers include amlodipine, aranidipine, barnidipine, benidipine, cilnidipine, clentiazem, diltiazen, efonidipine, fantofarone, felodipine, isradipine, lacidipine, lercanidipine, manidipine, mibefradil, nicardipine, nifedipine, nilvadipine, nisoldipine, nitrendipine, semotiadil, and veraparmil. The cholinesterase inhibitor and calcium channel blocker can be administered separately or in the form of a composition.

The invention provide methods for treating memory loss, cognitive impairments or dementia; and for treating and/or delaying the onset of Alzheimer's disease, vascular dementia, or dementia associated with Parkinson's disease by administering to a patient in need thereof at least one cholinesterase inhibitor and a therapeutically effective amount of caffeine. In another embodiment, the invention provides methods for treating and/or delaying the onset of Alzheimer's disease, vascular dementia, or dementia associated with Parkinson's' disease by administering to a patient in need thereof at least two cholinesterase inhibitors and a therapeutically effective amount of caffeine. The cholinesterase inhibitor and caffeine (or caffeine-containing compound) can be administered separately or in the form of a composition.

The invention provides methods for treating and/or delaying the onset of Alzheimer's disease or vascular dementia by administering to a patient in need thereof at least one cholinesterase inhibitor and at least one GABA inverse agonist. Any GABA inverse agonist in the art can be used. In one embodiment, the GABA inverse agonist is NGD 97-1. The cholinesterase inhibitor and GABA inverse agonist can be administered separately or in the form of a composition.

The invention provides methods for potentiating the effect of analgesics by administering to a patient in need thereof a therapeutically effective amount of at least one cholinesterase inhibitor and at least one analgesic. The use of a cholinesterase inhibitor in combination with the analgesic allows for the use of a lower dose of the analgesic to achieve the same results. The invention provides methods for treating one or more side-effects of analgesics by administering to a patient in need thereof a therapeutically effective amount of at least one cholinesterase inhibitor. The invention also provides methods for preventing emergence reactions (e.g., hallucinations) by administering to a patient in need thereof a therapeutically effective amount of at least one cholinesterase inhibitor and at least one narcotic. The analgesic can be any in the art. Exemplary analgesics include narcotics, NSAIDs, meperidine, propoxyphene and the like. The narcotic can be any in the art. Exemplary narcotics include alfentanil, allylprodine, alphaprodine, anileridine, benzylmorphine, bezitramide, buprenorphine, butorphanol, clonitazene, codeine, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazine, fentanyl, heroin, hydrocodone, hydromorphone, hydroxypethidine, isomethadone, ketobemidone, levallorphan, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normophine, norpipanone, opium, oxycodone, oxymorphone, papaveretum, pentazocine, phenadoxone, phenomorphan, phenazocine, phenoperidine, piminodine, piritramide, propheptazine, promedol, properidine, propiram, propoxyphene, sufentanil, tramadol, tilidine and the like. The MAID can be any in the art. Exemplary NSAIDs, include COX-1 and COX-2 inhibitors, such as celecoxib, rofecoxib, valdecoxib, ibuprofen, acetaminophen, aspirin, ketorolac, ketoprofen, diflunisal, salsalate, salicylates, salicylamide, thiosalicylates, mesalamine, sulfasalazine, methylsalicylate, phenylbutazone, oxyphenbutazone, antipyrine, aminopyrine, dipyrone, azapropazone, phenacetin, indomethacin, sulindac, mefenamic, meclofenamic, flufenamic, tolfenamic, etofenamic, tolmetin, naproxen, flurbiprofen, fenoprofen, fenbufen, pirprofen, oxaprozin, indoprofen, tiaprofenic acid, piroxicam, ampiroxicam, tenoxicam, tenidap, diclofenac, etodolac, nabumentone, and the like. The cholinesterase inhibitor and analgesic can be administered separately or in the form of a composition.

The invention provides methods for treating incontinence in a patient in need thereof by administering a therapeutically effective amount of at least one cholinesterase inhibitor. “Incontinence” is the inability to prevent the discharge of excretions, such as urine or feces. In one embodiment, the invention provides methods for treating urinary incontinence in a patient in need thereof by administering a therapeutically effective amount of at least one cholinesterase inhibitor. In one embodiment, the invention provides methods for treating fecal incontinence in a patient in need thereof by administering a therapeutically effective amount of at least one cholinesterase inhibitor.

The invention provides methods for treating constipation in a patient in need thereof by administering a therapeutically effective amount of at least one cholinesterase inhibitor.

The invention provides methods for treating wasting in a patient in need thereof by administering a therapeutically effective amount of at least one cholinesterase inhibitor. “Wasting” is the involuntary loss of more than 10% of body weight, and can also be accompanied by more than thirty days of either diarrhea, or weakness and fever. The critical component of weight loss in wasting is the loss of body cell mass. Body cell mass contains the metabolically active tissues of the body, including muscle cells, organ cells, and cells of the immune system. In wasting, the muscles waste away and the immune system is weakened. Wasting is often a problem for people living with AIDS (HIV disease) or cancer. Wasting can also be referred to as wasting syndrome.

The invention provides methods for treating or preventing chronic fatigue syndrome in a patient in need thereof by administering a therapeutically effective amount of at least one cholinesterase inhibitor. Chronic fatigue syndrome can be characterized by having severe chronic fatigue for six months or longer with other known medical conditions excluded by clinical diagnosis, and also having four or more of the following symptoms: substantial impairment in short-term memory or concentration, sore throat, tender lymph nodes, muscle pain, multi joint pain without swelling or redness, headaches of a new type, pattern or severity, unrefreshing sleep, and/or post-exertion malaise lasting more than 24 hours.

The invention also provides veterinary uses for cholinesterase inhibitors. In one embodiment, the invention provides methods for treating memory loss or anxiety in a patient in need thereof by administering an effective amount of at least one cholinesterase inhibitor and a veterinarily-acceptable carrier. The patient can be any mammal, such as cats and dogs. In another embodiment, the invention provides methods for treating excessive barking in a dog in need thereof by administering an effective amount of at least one cholinesterase inhibitor and a veterinarily-acceptable carrier.

The cholinesterase inhibitor used in the methods and compositions of the invention can be any in the art. The cholinesterase inhibitor can be, for example, an acetylcholinesterase inhibitor or a butyrylcholinesterase inhibitor. Acetylcholinesterase inhibitors are preferred. Exemplary cholinesterase inhibitors include donepezil, phenserine, tolserine, phenethylnorcymserine, ganstigmine, epastigmine, tacrine, physostigmine, pyridostigmine, neostigmine, rivastigmine, galantamine, citicoline, velnacrine, huperzine (e.g., huperzine A), metrifonate, heptastigmine, edrophonium, TAK-147 (i.e., 3-[1-(phenylmethyl)-4-piperidinyl]-1-(2,3,4,5-tetrahydro-1H-1-benzazepin-8-yl)-1-propanone fumarate or other salts thereof), T-82, upreazine, and the like. In each of the methods described herein, one or more cholinesterase inhibitors can be used. In one embodiment, one cholinesterase inhibitor is used. In another embodiment, donepezil, a stereoisomer thereof and/or a pharmaceutically acceptable salt thereof and a second cholinesterase inhibitor are used in the methods or compositions of the invention.

In one embodiment, the cholinesterase inhibitor can be a compound of formula I, a stereoisomer thereof, and/or a pharmaceutically acceptable salt thereof:

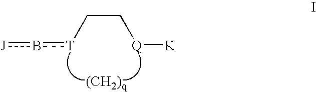

wherein J is
(a) a substituted or unsubstituted group selected from the group consisting of (1) phenyl, (2) pyridyl, (3) pyrazyl, (4) quinolyl, (5) cyclohexyl, (6) quinoxalyl, and (7) furyl;
(b) a monovalent or divalent group, in which the phenyl can have one or more substituents selected from (1) indanyl, (2) indanonyl, (3) indenyl, (4) indenonyl, (5) indanedionyl, (6) tetralonyl, (7) benzosuberonyl, (8) indanolyl, and (9) C6H5—CO—CH(CH3)—;
(c) a monovalent group derived from a cyclic amide compound;
(d) a lower alkyl group; or
(e) a group of R²¹—CH═CH—, in which R²¹ is hydrogen or a lower alkoxycarbonyl group;

B is —(CHR²²)r- , —CO—(CHR²²)r-, —NR4-(CHR²²)r-, —CO—NR⁵—(CHR²²)r-, —CH═CH—(CHR²²)—, —OCOO—(CHR²²)r-, —OOC—NH—(CHR²²)r-, —NH—CO—(CHR²²)r-, —CH₂—CO—NH—(CHR²²)r-, —(CH₂)₂—NH—(CHR²²)r-, —CH(OH)—(CHR²²)r-, ═(CH—CH═CH)_{b}—, ═CH—(CH₂)_{c}—, ═(CH—CH)_{d}═, —CO—CH═CH—CH₂—, —CO—CH₂—CH(OH)—CH₂—, —CH(CH₃)—CO—NH—CH₂—, —CH═CH═CO—NH—(CH₂)₂—, —NH—, —O—, —S—, a dialkylaminoalkyl-carbonyl or a lower alkoxycarbonyl;

wherein R⁴ is hydrogen, lower alkyl, acyl, lower alkylsulfonyl, phenyl, substituted phenyl, benzyl, or substituted benzyl; R⁵ is hydrogen, lower alkyl or phenyl; r is zero or an integer of about 1 to about 10; R²² is hydrogen or methyl so that one alkylene group can have no methyl branch or one or more methyl branches; b is an integer of about 1 to about 3; c is zero or an integer of about 1 to about 9; d is zero or an integer of about 1 to about 5;

T is nitrogen or carbon;

Q is nitrogen, carbon or

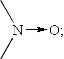

q is an integer of about 1 to about 3;

K is hydrogen, phenyl, substituted phenyl, arylalkyl in which the phenyl can have a substituent, cinnamyl, a lower alkyl, pyridylmethyl, cycloalkylalkyl, adamantanemethyl, furylmenthyl, cycloalkyl, lower alkoxycarbonyl or an acyl; and

[symbol] is a single bond or a double bond.

In the compound of formula 1, J is preferably (a) or (b), more preferably (b). In the definition of (b), a monovalent group (2), (3) and (5) and a divalent group (2) are preferred. The group (b) preferably includes, for example, the groups having the formulae shown below:

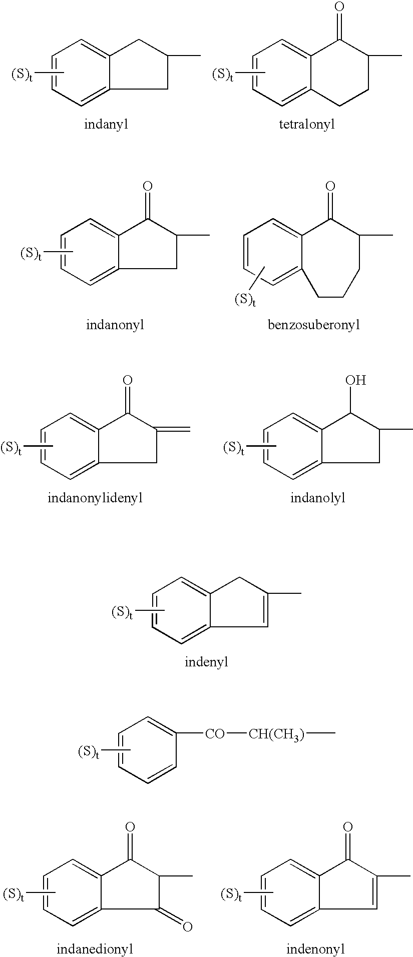

wherein t is an integer of about 1 to about 4; and each S is independently hydrogen or a substituent, such as a lower alkyl having 1 to 6 carbon atoms or a lower alkoxy having 1 to 6 carbon atoms. Among the substituents, methoxy is most preferred. The phenyl is most preferred to have 1 to 3 methoxy groups thereon. (S)ₜ can form methylene dioxy groups or ethylene dioxy groups on two adjacent carbon atoms of the phenyl group. Of the above groups, indanonyl, indanedionyl and indenyl, optionally having substituents on the phenyl, are the most preferred.

In the definition of B, —(CHR²²)ᵣ—, —CO—(CHR²²)ᵣ—, ═(CH—CH═CH)_{b}—, ═CH—(CH₂)_{c}— and ═(CH—CH)_{d}=are preferable. The group of —(CHR²²)r- in which R²² is hydrogen and r is an integer of 1 to 3, and the group of ═CH—(CH₂)c- are most preferable. The preferable groups of B can be connected with (b) of J, in particular (b)(2).

The ring containing T and Q in formula I can be 5-, 6- or 7-membered. It is preferred that Q is nitrogen, T is carbon or nitrogen, and q is 2; or that Q is nitrogen, T is carbon, and q is 1 or 3; or that Q is carbon, T is nitrogen and q is 2.

It is preferable that K is a phenyl, arylalkyl, cinnamyl, phenylalkyl or a phenylalkyl having a substituent(s) on the phenyl.

In another embodiment, the cyclic amine compounds of formula I are the piperidine compounds of formula 11, a stereoisomer thereof, and/or a pharmaceutically acceptable salt thereof:

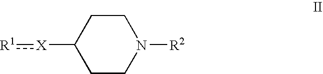

wherein R¹ is a (1) substituted or unsubstituted phenyl group; (2) a substituted or unsubstituted pyridyl group; (3) a substituted or unsubstituted pyrazyl group; (4) a substituted or unsubstituted quinolyl group; (5) a substituted or unsubstituted indanyl group; (6) a substituted or unsubstituted cyclohexyl group; (7) a substituted or unsubstituted quinoxalyl group; (8) a substituted or unsubstituted furyl group; (9) a monovalent or divalent group derived from an indanone having a substituted or unsubstituted phenyl ring; (10) a monovalent group derived from a cyclic amide compound; (11) a lower alkyl group; or (12) a group of the formula R³—CH═C—, where R³ is a hydrogen atom or a lower alkoxycarbonyl group;

X is —(CH₂)ₙ—, —C(O)—(CH₂)ₙ—, —N(R⁴)—(CH₂)ₙ—, —C(O)—N(R⁵)—(CH₂)ₙ—, —CH═CH—(CH₂)ₙ—, —O—C(O)—O—(CH₂)ₙ—, —O—C(O)—NH—(CH₂)ₙ—, —CH═CH—CH═CO—, —NH—C(O)—(CH₂)ₙ—, —CH₂—C(O)—NH—(CH₂)ₙ—, —(CH₂)₂—C(O)—NH—(CH₂)ₙ—, —CH(OH)—(CH₂)ₙ—, —C(O)—CH═CH—CH₂—, —C(O)—CH₂—CH(OH)—CH₂—, —CH(CH₃)—C(O)—NH—CH₂—, —CH═CH—C(O)—NH—(CH₂)₂—, a dialkylaminoalkylcarbonyl group, a lower alkoxycarbonyl group;

where n is an integer of 0 to 6; R⁴ is a hydrogen atom, a lower alkyl group, an acyl group, a lower alkylsulfonyl group, a substituted or unsubstituted phenyl group, or a substituted or unsubstituted benzyl group; and R⁵ is a hydrogen atom a lower alkyl group or a phenyl group;

R2 is a substituted or unsubstituted phenyl group; a substituted or unsubstituted arylalkyl group; a cinnamyl group; a lower alkyl group; a pyridylmethyl group; a cycloalkylalkyl group; an adamantanemethyl group; or a furoylmethyl group; and

[symbol] is a single bond or a double bond.

The term “lower alkyl group” as used herein means a straight or branched alkyl group having 1 to 6 carbon atoms. Exemplary “lower alkyl groups” include methyl, ethyl, propyl, isopropyl, butyl, isobutyl, sec-butyl, tert-butyl, pentyl (amyl), isopentyl, neopentyl, tert-pentyl, 1-methylbutyl, 2-methylbutyl, 1,2-dimethylpropyl, hexyl, isohexyl, 1-methylpentyl, 2-methyl-pentyl, 3-methylpentyl, 1,1-dimethylbutyl, 1,2-dimethylbutyl, 2,2-dimethylbutyl, 1,3-dimthyl-butyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, and the like. The lower alkyl group is preferably methyl, ethyl, propyl or isopropyl; more preferably methyl.

Specific examples of the substituents for the substituted or unsubstituted phenyl, pyridyl, pyrazyl, quinolyl, indanyl, cyclohexyl, quinoxalyl and furyl groups in the definition of R¹ include lower alkyl groups having 1 to 6 carbon atoms, such as methyl, ethyl, n-propyl, isopropyl, n-butyl, isobutyl, and tert-butyl groups; lower alkoxy groups corresponding to the above-described lower alkyl groups, such as methoxy and ethoxy groups; a nitro group; halogen atoms, such as chlorine, fluorine and bromine; a carboxyl group; lower alkoxycarbonyl groups corresponding to the above-described lower alkoxy groups, such as methoxycarbonyl, ethoxycarbonyl, isopropoxycarbonyl, n-propoxycarbonyl, and n-butyloxycarbonyl groups; an amino group; a lower monoalkylamino group; a lower dialkylamino group; a carbamoyl group; acylamino groups derived from aliphatic saturated monocarboxylic acids having 1 to 6 carbon atoms, such as acetylamino, propionylamino, butyrylamino, isobutyrylamino, valerylamino, and pivaloylamino groups; cycloalkyoxycarbonyl groups, such as a cyclohexyloxycarbonyl group; lower alkylaminocarbonyl groups, such as methylaminocarbonyl and ethylaminocarbonyl groups; lower alkylcarbonyloxy groups corresponding to the above-defined lower alkyl groups, such as methylcarbonyloxy, ethylcarbonyloxy, and n-propylcarbonyloxy groups; halogenated lower alkyl groups, such as a trifluoromethyl group; a hydroxyl group; a formyl group; and lower alkoxy lower alkyl groups, such as ethoxymethyl, methoxymethyl and methoxyethyl groups. The “lower alkyl groups” and “lower alkoxy groups” in the above description of the substituent include all the groups derived from the above-mentioned groups. The substituent can be one to three of them, which can be the same or different.

When the substituent is a phenyl group, the following group is within the scope of the substituted phenyl group:

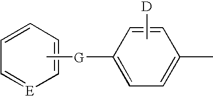

wherein G is —C(O)—, —O—C(O)—, —O—, —CH₂—NH—C(O)—, —CH₂—O—, —CH₂—SO₂—, —CH(OH)—, or —CH₂—S(→O)—; E is a carbon or nitrogen atom; and D is a substituent. Preferred examples of the substituents (i.e., “D”) for the phenyl group include lower alkyl, lower alkoxy, nitro, halogenated lower alkyl, lower alkoxycarbonyl, formyl, hydroxyl, and lower alkoxy lower alkyl groups, halogen atoms, and benzyol and benzylsulfonyl groups. The substituent can be two or more of them, which can be the same or different.

Preferred examples of the substituent for the pyridyl group include lower alkyl and amino groups and halogen atoms.

Preferred examples of the substituent for the pyrazyl group include lower alkoxycarbonyl, carboxyl, acylamino, carbamoyl, and cycloalkyoxycarbonyl groups. With respect to R¹, the pyridyl group is preferably a 2-pyridyl, 3-pyridyl, or 4-pyridyl group; the pyrazyl group is preferably a 2-pyrazinyl group; the quinolyl group is preferably a 2-quinolyl or 3-quinolyl group; the quinoxalinyl group is preferably a 2-quinoxalinyl or 3-quinoxalinyl group; and the furyl group is preferably a 2-furyl group.

Specific examples of preferred monovalent or divalent groups derived from an indanone having an unsubstituted or substituted phenyl ring include those represented by formulas (A) and (B):

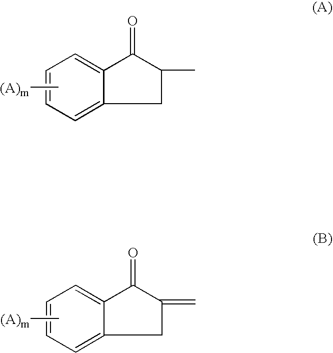

where m is an integer of from 1 to 4, and each A is independently a hydrogen atom, a lower alkyl group, a lower alkoxy group, a nitro group, a halogen atom, a carboxyl group, a lower alkoxycarbonyl group, an amino group, a lower monoalkylamino group, a lower dialkylamino group, a carbamoyl group, an acylamino group derived from aliphatic saturated monocarboxylic acids having 1 to 6 carbon atoms, a cycloalkyloxycarbonyl group, a lower alkylaminocarbonyl group, a lower alkylcarbonyloxy group, a halogenated lower alkyl group, a hydroxyl group, a formyl group, or a lower alkoxy lower alkyl group; preferably a hydrogen atom, a lower alkyl group or a lower alkoxy group; most preferably the indanone group is unsubstituted or substituted with 1 to 3 methoxy groups.

Examples of the monovalent group derived from a cyclic amide compound include quinazolone, tetrahydroisoquinolinone, tetrahydrobenzodiazepinone, and hexahydrobenzazocinone. However, the monovalent group can be any one having a cyclic amide group in the structural formula thereof, and is not limited to the above-described specific examples. The cyclic amide group can be one derived from a monocyclic or condensed heterocyclic ring. The condensed heterocyclic ring is preferably one formed by condensation with a phenyl ring. In this case, the phenyl ring can be substituted with a lower alkyl group having 1 to 6 carbon atoms, preferably a methyl group, or a lower alkoxy group having 1 to 6 carbon atoms, preferably a methoxy group.

Preferred examples of the monovalent group include the following:

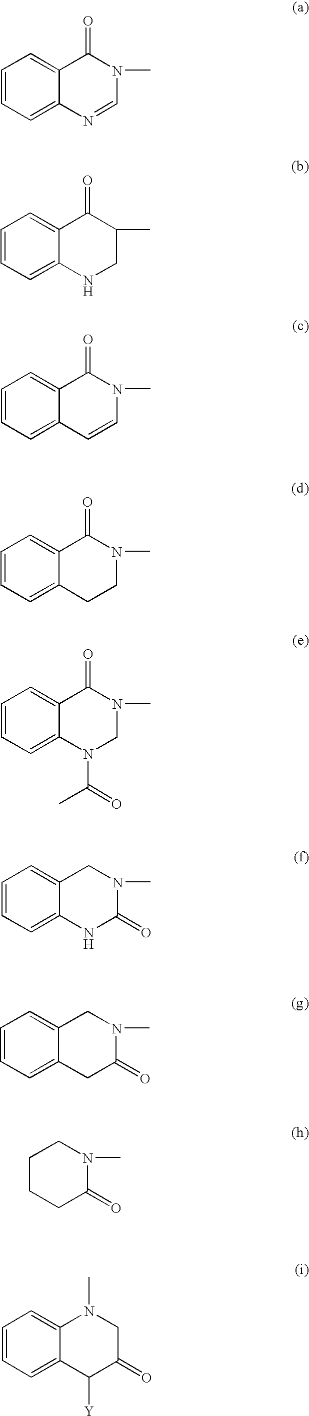

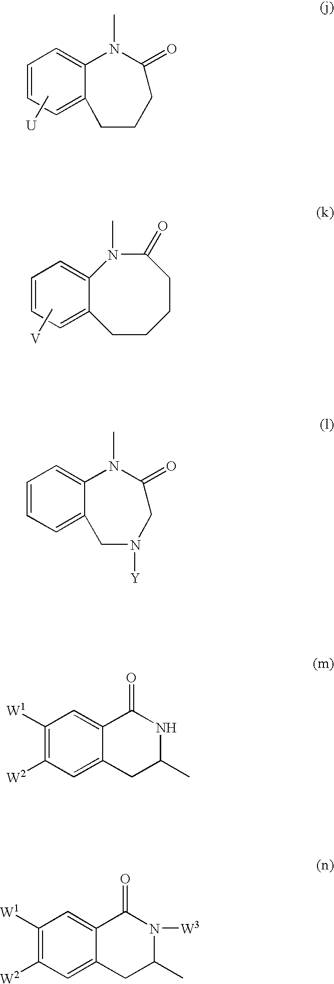

In the above formulae, Y is a hydrogen atom or a lower alkyl group; V and U are each a hydrogen atom or a lower alkoxy group (preferably dimethoxy); W¹ and W² are each a hydrogen atom, a lower alkyl group, or a lower alkoxy group; and W³ is a hydrogen atom or a lower alkyl group. The right hand ring in formulae (j) and (l) is a 7-membered ring, while the right hand ring in formula (k) is an 8-membered ring.

The most preferred examples of the above-defined R¹ include a monovalent group derived from an indanone having an unsubstituted or substituted phenyl group and a monovalent group derived from a cyclic amide compound.

The most preferred examples of the above-defined X include —(CH₂)ₙ—, an amide group, or groups represented by the above formulae where n is 2. Thus, it is most preferred that any portion of a group represented by the formula R¹ [symbol] X- have a carbonyl or amide group.

The substituents involved in the expressions “a substituted or unsubstituted phenyl group” and “a substituted or unsubstituted arylalkyl group” in the above definition of R2 are the same substituents as those described for the above definitions of a phenyl group, a pyridyl group, a pyrazyl group, a quinolyl group, an indanyl group, a cyclohexyl group, a quinoxalyl group or a furyl group in the definition of R¹.

The term “arylalkyl group” is intended to mean an unsubstituted benzyl or phenethyl group or the like.

Specific examples of the pyridylmethyl group include 2-pyridylmethyl, 3-pyridylmethyl, and 4-pyridylmethyl groups.

Preferred examples of R2 include benzyl and phenethyl groups. The symbol [symbol] means a double or single bond. The bond is a double bond only when R¹ is the divalent group (B) derived from an indanone having an unsubstituted or substituted phenyl ring, while it is a single bond in other cases.

In another embodiment, the compound of formula II is a compound of formula III, a stereoisomer thereof, and/or a pharmaceutically acceptable salt thereof:

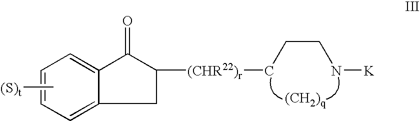

wherein r is an integer of about 1 to about 10; each R²² is independently hydrogen or methyl; K is a phenalkyl or a phenalkyl having a substituent on the phenyl ring; each S is independently a hydrogen, a lower alkyl group having 1 to 6 carbon atoms or a lower alkoxy group having 1 to 6 carbon atoms; t is an integer of 1 to 4; q is an integer of about 1 to about 3; with the proviso that (S)ₜ can be a methylenedioxy group or an ethylenedioxy group joined to two adjacent carbon atoms of the phenyl ring.

In other embodiments, the compound of formula III is 1-benzyl-4-((5,6-dimethoxy-1-indanon)-2-yl)methylpiperidine; 1-benzyl-4-((5,6-dimethoxy-1-indanon)-2-ylidenyl)methylpiperidine; 1-benzyl-4-((5-methoxy-1-indanon)-2-yl)methylpiperidine; 1-benzyl-4-((5,6-diethoxy-1-indanon)-2-yl)methylpiperidine; 1-benzyl-4-((5,6-methnylenedioxy-1-indanon)-2-yl)methylpiperidine; 1-(m-nitrobenzyl)-4-((5,6-dimethoxy-1-indanon)-2-yl)methylpiperidine; 1-cyclohexylmethyl-4-((5,6-dimethoxy-1-indanon)-2-yl)methylpiperidine; 1-(m-fluorobenzyl)-4-((5,6-dimethoxy-1-indanon)-2-yl)methylpiperidine; 1-benzyl-4-((5,6-dimethoxy-1-indanon)-2-yl)propylpiperidine; 1-benzyl-4-((5-isopropoxy-6-methoxy-1-indanon)-2-yl)methylpiperidine; 1-benzyl-4-((5,6-dimethoxy-1-oxoindanon)-2-yl)propenylpiperidine; or a stereoisomer and/or a pharmaceutically acceptable salt thereof.

In still other embodiments, the compound of formula III is 1-benzyl-4-((5,6-dimethoxy-1-indanon)-2-yl)methylpiperidine, a stereoisomer thereof and/or a pharmaceutically acceptable salt thereof, which is represented by formula IV:

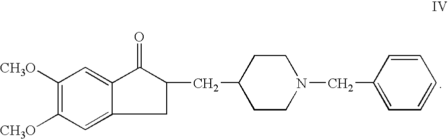

In still other embodiments, the compound of formula III is 1-benzyl-4-((5,6-dimethoxy-1-indanon)-2-yl)methylpiperidine hydrochloride or a stereoisomer thereof, which is also known as donepezil hydrochloride or ARICEPT® (Eisai Inc., Teaneck, N.J.), and which is represented by formula IVa:

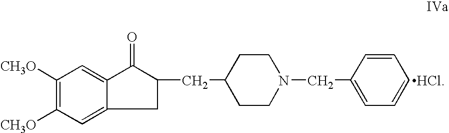

The compounds of the invention can have an asymmetric carbon atom(s), depending upon the substituents, and can have stereoisomers, which are within the scope of the invention. For example, donepezil or pharmaceutically acceptable salts thereof can be in the forms described in Japanese Patent Application Nos. 4-187674 and 4-21670, the disclosures of which are incorporated by reference herein in their entirety.

Japanese Patent Application No. 4-187674 describes a compound of formula V:

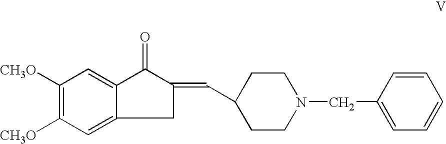

which can be in the form of a pharmaceutically acceptable salt, such as a hydrochloride salt. Japanese Patent Application No. 4-21670 describes compounds of formula VI:

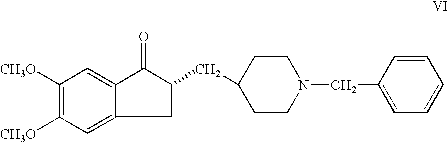

which can be in the form of a pharmaceutically acceptable salt, such as a hydrochloride salt; and compounds of formula VII:

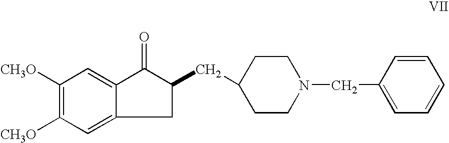

which can be in the form of a pharmaceutically acceptable salt, such as a hydrochloride salt; and compounds of formula VIII:

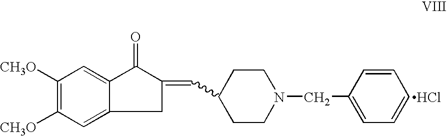

As described above, the cholinesterase inhibitors and other medications described herein can be administered in the form of a pharmaceutically acceptable salt. Pharmaceutically acceptable salts are well known in the art and include those of inorganic acids, such as hydrochloride, sulfate, hydrobromide and phosphate; and those of organic acids, such as formate, acetate, trifluoroacetate, methanesulfonate, benzenesulfonate and toluenesulfonate. When certain substituents are selected, the compounds of the invention can form, for example, alkali metal salts, such as sodium or potassium salts; alkaline earth metal salts, such as calcium or magnesium salts; organic amine salts, such as a salt with trimethylamine, triethylamine, pyridine, picoline, dicyclohexylamine or N,N′-dibenzylethylenediamine. One skilled in the art will recognize that the compounds of the invention can be made in the form of any other pharmaceutically acceptable salt.

The cholinesterase inhibitors can be prepared by processes that are known in the art and described, for example, in U.S. Pat. No. 4,895,841, WO 98/39000, and Japanese Patent Application Nos. 4-187674 and 4-21670, the disclosures of each of which are incorporated by reference herein in their entirety. Donepezil hydrochloride, a preferred cholinesterase inhibitor for use in the methods described herein, is commercially available as ARICEPT® from Eisai Inc., Teaneck, N.J. The other medications described herein (e.g., statins, vaccines, anti-oxidants, psychiatric medications, NMDA receptor blockers, calcium channel blockers, caffeine, analgesics, GABA inverse agonists) are commercially available, are in development, and/or can be prepared by processes described in the literature. The cholinesterase inhibitors and other medications described herein can be administered as pharmaceutical combinations. A pharmaceutical combination is a pharmaceutical formulation comprising both active ingredients or separate pharmaceutical dosage forms.

The dosage regimen for treating and preventing the diseases described herein with the cholinesterase inhibitors and other medications can be selected in accordance with a variety of factors, including the age, weight, sex, and medical condition of the patient, the severity of the migraines, the route of administration, pharmacological considerations such as the activity, efficacy, pharmacokinetic and toxicology profiles of the drugs, whether a drug delivery system is used and whether the cholinesterase inhibitor is administered as part of a drug combination.

When more than one cholinesterase inhibitor is administered to a patient and/or when the cholinesterase inhibitor(s) is administered in conjunction with another medication, the compounds can be separately administered about the same time as part of an overall treatment regimen, i.e., as a drug cocktail or combination therapy. “About the same time” includes administering the compounds at the same time, at different times on the same day, or on different days, as long as they are administered as part of an overall treatment regimen.

The cholinesterase inhibitors can be administered to treat or prevent the diseases described herein in doses of about 0.01 milligrams to about 300 milligrams per day, preferably about 1 milligram to about 100 milligrams per day, more preferably about 5 milligrams to about 10 milligrams per day. The doses can be administered in one to four portions over the course of a day, preferably once a day. One skilled in the art will recognize that when the cholinesterase inhibitors are administered to children, the dose can be smaller than the dose administered to adults, and that the dose can be dependent upon the size and weight of the patient. A child can be administered the cholinesterase inhibitors in doses of about 0.1 milligrams to about 15 milligrams per day, preferably about 0.5 milligrams to about 10 milligrams per day, more preferably about 1 milligram to about 3 milligrams per day.

In other embodiments of the methods described herein, donepezil hydrochloride, which is commercially available as ARICEPT® (Eisai Inc., Teaneck, N.J.), can be administered as tablets containing either 5 milligrams donepezil hydrochloride or 10 milligrams donepezil hydrochloride. The tablets can be administered one to about four times a day. In preferred embodiments, one 5 milligram or one 10 milligram ARICEPT® tablet is administered once a day for the methods described herein. One skilled in the art will appreciate that when donepezil hydrochloride is administered to children, the dose can be smaller than the dose that is administered to adults.

The cholinesterase inhibitors and other medications of the invention can be administered orally, topically, parenterally, by inhalation (nasal or oral), or rectally in dosage unit formulations containing conventional nontoxic pharmaceutically acceptable carriers, adjuvants, and vehicles as desired. The term parenteral includes subcutaneous, intravenous, intramuscular, intrathecal, intrastemal injection, or infusion techniques. Preferably, the cholinesterase inhibitors are orally administered as tablets. When administered to children, the cholinesterase inhibitors are preferably orally administered in a liquid dosage form.

Injectable preparations, for example, sterile injectable aqueous or oleaginous suspensions, of the cholinesterase inhibitors can be formulated according to the art using suitable dispersing or wetting agents, suspending agents (e.g., methylcellulose, Polysorbate 80, hydroxyethylcellulose, acacia, powdered tragacanth, sodium carboxymethylcellulose, polyoxytehylene sorbitan monolaurate and the like), pH modifiers, buffers, solubilizing agents (e.g., polyoxyethylene hydrogenated castor oil, Polysorbate 80, nicotinamide, polyoxyethylene sorbitan monolaurate, Macrogol, an ethyl ester of castor oil fatty acid, and the like) and preservatives. The sterile injectable preparation can also be a sterile injectable solution or suspension in a nontoxic parenterally acceptable diluent or solvent, for example, as a solution in 1,3-butanediol. Among the acceptable vehicles and solvents that can be used are water, Ringer's solution, and isotonic sodium chloride solution. In addition, sterile, fixed oils are conventionally used as a solvent or suspending medium. For this purpose any bland fixed oil can be used including synthetic mono- or diglycerides, in addition, fatty acids, such as oleic acid, can be used in the preparation of injectables. The preparations can be lyophilized by methods known in the art.

Solid dosage forms for oral administration of the cholinesterase inhibitors can include chewing gum, capsules, tablets, sublingual tablets, powders, granules, and gels; preferably tablets. In such solid dosage forms, the active compound can be admixed with one or more inert diluents such as lactose or starch. As is normal practice, such dosage forms can also comprise other substances including lubricating agents such as magnesium stearate. In the case of capsules, tablets, and pills, the dosage forms can also comprise buffering agents. The tablets can be prepared with enteric or film coatings, preferably film coatings.

Sublingual administration refers to the administration of the cholinesterase inhibitors in the mouth (e.g., under the tongue, between the cheek and gum, between the tongue and roof of the mouth). The highly vascular mucosal lining in the mouth is a convenient location for the cholinesterase inhibitors to be administered into the body. To make tablets, the cholinesterase inhibitors can be admixed with pharmaceutically acceptable carriers known in the art such as, for example, vehicles (e.g., lactose, white sugar, mannitol, glucose, starches, calcium carbonate, crystalline cellulose, silicic acid, and the like), binders (e.g., water, ethanol, myranol, glucose solution, starch solution, gelatin solution, polyvinylpyrrolidone, and the like), disintegrators (e.g., dry starch, sodium, alginate, sodium hydrogen carbonate, calcium carbonate, polyoxyethylene sorbitan fatty acid esters, sodium laurylsulfate, stearic monoglyceride, starches, lactose, and the like), absorption promoters (e.g., quaternary ammonium base, sodium laurylsulfate, and the like), wetting agents (e.g. glycerin, starches, and the like), lubricants (e.g., stearates, polyethylene glycol, and the like), and flavoring agents (e.g., sweeteners). The tablets can be in the form of a conventional tablet, a molded tablet, a wafer and the like.

In other embodiments, the solid dosage form can be packaged as granules or a powder in a pharmaceutically acceptable carrier, where the granules or powder are removed from the packaging and sprinkled on food or mixed with a liquid, such as water or juice. In this embodiment, the cholinesterase inhibitors can be mixed with flavoring or sweetening agents. The packaging material can be plastic, coated paper, or any material that prevents water or moisture from reaching the granules and/or powder.

Liquid dosage forms for oral administration of the cholinesterase inhibitors can include pharmaceutically acceptable emulsions, solutions, sublingual solutions, suspensions, and syrups containing inert diluents commonly used in the art, such as water. Such compositions can also comprise adjuvants, such as wetting agents, emulsifying and suspending agents, and sweetening, flavoring, and perfuming agents. To make sublingual solutions, the cholinesterase inhibitors can be admixed with various carriers, excipients, pH adjusters, and the like (e.g., water, sugar, lactic acid, acetic acid, fructose, glucose, saccharin, polyethylene glycol, propylene glycol, alcohol, bentonite, tragacanth, gelatin, alginates, aspartame, sorbitol, methylparaben, propylparaben, sodium benzoate, artificial flavoring and coloring agents).

For administration by inhalation, the cholinesterase inhibitors can be delivered from an insufflator, a nebulizer or a pressured pack or other convenient mode of delivering an aerosol spray. Pressurized packs can include a suitable propellant. Alternatively, for administration by inhalation, the cholinesterase inhibitors can be administered in the form of a dry powder composition or in the form of a liquid spray.

Suppositories for rectal administration of the cholinesterase inhibitors can be prepared by mixing the active compounds with suitable nonirritating excipients such as cocoa butter and polyethylene glycols that are solid at room temperature and liquid at body temperature. Alternatively, an enema can be prepared by for rectal administration of the cholinesterase inhibitors.

For topical administration to the epidermis, the cholinesterase inhibitors can be formulated as ointments, creams or lotions, or as the active ingredient of a transdermal patch. The cholinesterase inhibitors can also be administered via iontophoresis or osmotic pump. Ointments, creams and lotions can be formulated with an aqueous or oily base with the addition of suitable thickening and/or gelling agents. Alternatively, ointments, creams and lotions can be formulated with an aqueous or oily base and can also contain one or more emulsifying agents, stabilizing agents, dispersing agents, suspending agents, thickening agents, and/or coloring agents. As creams or lotions, the cholinesterase inhibitors can be mixed to form a smooth, homogeneous cream or lotion with, for example, one or more of a preservative (e.g., benzyl alcohol 1% or 2% (wt/wt)), emulsifying wax, glycerin, isopropyl palmitate, lactic acid, purified water, sorbitol solution. Such topically administrable compositions can contain polyethylene glycol 400. To form ointments, the cholinesterase inhibitors can be mixed with one or more of a preservative (e.g., benzyl alcohol 2% (wt/wt)), petrolatum, emulsifying wax, and Tenox (II) (e.g., butylated hydroxyanisole, propyl gallate, citric acid, propylene glycol). Woven pads or rolls of bandaging material, e.g., gauze, can be impregnated with the transdermally administrable compositions for topical application.

The cholinesterase inhibitors can also be topically applied using a transdermal system, such as one of an acrylic-based polymer adhesive with a resinous crosslinking agent impregnated with the cholinesterase inhibitors and laminated to an impermeable backing. For example, the cholinesterase inhibitors can be administered in the form of a transdermal patch, such as a sustained-release transdermal patch. Transdermal patches can include any conventional form such as, for example, an adhesive matrix, a polymeric matrix, a reservoir patch, a matrix- or monolithic-type laminated structure, and are generally comprised of one or more backing layers, adhesives, penetration enhancers, and/or rate-controlling membranes. Transdermal patches generally have a release liner which is removed to expose the adhesive/active ingredient(s) prior to application. Transdermal patches are described in, for example, U.S. Pat. Nos. 5,262,165, 5,948,433, 6,010,715 and 6,071,531, the disclosures of which are incorporated by reference herein in their entirety.

For the methods of treating and/or preventing, for example, Sjogren's syndrome (e.g., with respect to problems associated with the eyes), neuronal loss in the eyes, and/or macular degeneration, the cholinesterase inhibitors can be topically administered to the affected eye(s) in the form of an ophthalmic composition. The cholinesterase inhibitors can be administered in doses of about 0.00001 milligrams to about 300 milligrams per day to the affected eye(s), preferably about 0.0001 milligrams to about 100 milligrams per day, more preferably about 0.0001 milligrams to about 10 milligrams per day. The doses can be administered in one to four portions over the course of a day, preferably once per day.

When administered in the form of eye drops, the cholinesterase inhibitors can be topically administered to the patient's eye in a concentration of about 0.00001% (e.g., 0.01 milligram cholinesterase inhibitor per 100 ml H2O) to about 1% (e.g., 1 gram cholinesterase inhibitor per 100 ml H20); preferably in a concentration of about 0.0001% to about 0.25%, and even more preferably in a concentration of about 0.001% to about 0.125%, most preferably in a concentration of about 0.01% to about 0.1%. One to four drops can be administered to the patient's eye(s) over the course of a day, preferably one or two drops per day, most preferably one drop per day. In yet another embodiment, the cholinesterase inhibitors can be administered in a concentration of about 0.125% to about 0.25% administered in one or two drops daily, preferably one drop daily.

The cholinesterase inhibitors can be topically administered to the affected eye(s) in the form of an ophthalmic composition. The ophthalmic composition can be in the form of a gel, a solution, a suspension, an emulsion (dispersion), or an erodible solid ocular insert. The ophthalmic compositions can comprise liposomes or microbubbles. Alternatively, the ophthalmic compositions can be administered in the form of non-aqueous formulations, such as substantially non-aqueous liquids, substantially non-aqueous semi-solid compositions, and solid compositions or devices. The methods of treating or preventing glaucoma and/or intraocular pressure typically comprise the topical application of one or two drops (or an equivalent amount of a solid or semi-solid dosage form) to the affected eye one to four times per day, preferably once per day.

In forming compositions for topical administration, the ophthalmic compositions are generally formulated at a pH between about 4.5 and about 8.0. The topical compositions can also comprise other ingredients that are known to be used in ophthalmic compositions including, for example, preservatives, surfactants and co-solvents, tonicity agents, and viscosity building agents.

Ophthalmic compositions are typically packaged in multidose form, which generally requires the addition of preservatives to prevent microbial contamination during use. Suitable preservatives include, for example, benzalkonium chloride, thimerosal, chlorobutanol, methyl paraben, propyl paraben, phenylethyl alcohol, edetate disodium, sorbic acid, ONAMER M®, and the like. Such preservatives are typically used at a concentration of about 0.001% to about 1.0% by weight.

Surfactants or co-solvents that can be used in the ophthalmic compositions of the invention include polysorbate 20, polysorbate 60, polysorbate 80, Pluronic F-68, Pluronic F-84, Pluronic P-103, TYLOXAPOL®, CREMOPHOR® EL, sodium dodecyl sulfate, glycerol, PEG 400, propylene glycol, cyclodextrins, and the like. Surfactants or co-solvents are typically used at a concentration of about 0.01% to about 2% by weight.

A viscosity greater than that of simple aqueous solutions can be desirable to increase ocular absorption of the active compounds, to decrease variability in dispensing the compositions, to decrease physical separation of components of a suspension or emulsion of the compositions and/or to otherwise improve the ophthalmic composition. Viscosity building agents include polyvinyl alcohol, polyvinyl pyrrolidone, methyl cellulose, hydroxy propyl methylcellulose, hydroxyethyl cellulose, carboxymethyl cellulose, hydroxy propyl cellulose and the like. Viscosity building agents are typically used at a concentration of about 0.01% to about 2% by weight.

Suitable agents which can be used to adjust the tonicity or osmolality of the ophthalmic compositions include sodium chloride, potassium chloride, mannitol, dextrose, glycerin, propylene glycol and the like. Tonicity agents are typically used at a concentration of about 0.1% to about 10% by weight.

Substantially non-aqueous liquids comprise at least one of the cholinesterase inhibitors of the invention dissolved or suspended in one or more of the following: vegetable and mineral oils, such as liquid petrolatum, corn oil, castor oil, sesame oil and peanut oil; triglycerides, such as the capric/caprylic triglycerides commonly used in foods and cosmetics; liquid lanolin and lanolin derivatives; and perfluorohydrocarbons.

Substantially non-aqueous semi-solid compositions comprise at least one of the cholinesterase inhibitors of the invention dissolved or suspended in or more of the following: various types of petrolatum, such as white, yellow and red; lanolin and lanolin derivatives; gelled mineral oil having a hydrocarbon base, such as PLASTIBASE®; petrolatum and ethylene carbonate mixtures; petrolatum in combination with surfactants and polyglycol, such as polyoxyl 40 stearate and polyethylene glycol.

Solid compositions or devices include non-erodible devices which are inserted into the conjunctival sac of the eye and later removed, such as the Alza-type diffusion or osmotic pressure controlled polymer membranes; and bioerodible polymers which do not have to be removed from the conjunctival sac, such as essentially anhydrous but water soluble polymers and resins (e.g., celluloses, polycarboxylic acids).

The invention provides for the cholinesterase inhibitors to be administered nasally to a patient to treat the diseases and disorders described herein and those described, for example, in PCT/US02/29734, WO 01/66114, and U.S. Pat. Nos. 6,482,838, 6,458,807 and 6,455,544, the disclosures of which are incorporated by reference herein in their entirety. “Administered nasally” or “nasal administration” is intended to mean that at least one cholinesterase inhibitor is combined with a suitable delivery system for absorption across the nasal mucosa of a patient, preferably a human. Generally, lower doses of the cholinesterase inhibitor can be used for nasal administration when compared, for example, to the dose required for the oral administration of the cholinesterase inhibitor.

The cholinesterase inhibitors of the invention can be administered, for example, as nasal sprays, nasal drops, nasal suspensions, nasal gels, nasal ointments, nasal creams or nasal powders. The cholinesterase inhibitors can also be administered using nasal tampons or nasal sponges. The cholinesterase inhibitors of the invention can be brought into a viscous basis via systems conventionally used, for example, natural gums, methylcellulose and derivatives, acrylic polymers (carbopol) and vinyl polymers (polyvinylpyrrolidone). In the compositions, many other excipients known in the art can be added such as water, preservatives, surfactants, solvents, adhesives, antioxidants, buffers, bio-adhesives, viscosity enhancing agents and agents to adjust the pH and the osmolarity.

The nasal delivery systems can take various forms including aqueous solutions, non-aqueous solutions and combinations thereof. Aqueous solutions include, for example, aqueous gels, aqueous suspensions, aqueous liposomal dispersions, aqueous emulsions, aqueous microemulsions and combinations thereof. Non-aqueous solutions include, for example, non-aqueous gels, non-aqueous suspensions, non-aqueous liposomal dispersions, non-aqueous emulsions, non-aqueous microemulsions and combinations thereof.

In other embodiments, the nasal delivery system can be a powder formulation. Powder formulations include, for example, powder mixtures, powder microspheres, coated powder microspheres, liposomal dispersions and combinations thereof. Preferably, the powder formulation is powder microspheres. The powder microspheres are preferably formed from various polysaccharides and celluloses selected from starch, methylcellulose, xanthan gum, carboxymethylcellulose, hydroxypropyl cellulose, carbomer, alginate polyvinyl alcohol, acacia, chitosans, and mixtures of two or more thereof.

In certain embodiments, the particle size of the droplets of the aqueous and/or non-aqueous solution or of the powders delivered to the nasal mucosa can be, for example, about 0.1 micron to about 100 microns; from about 1 micron to about 70 microns; from about 5 microns to about 50 microns; or from about 10 microns to about 20 microns. The particle sizes can be obtained using suitable containers or metering devices known in the art. Exemplary devices include mechanical pumps in which delivery is made by movement of a piston; compressed air mechanisms in which delivery is made by hand pumping air into the container; compressed gas (e.g., nitrogen) techniques in which delivery is made by the controlled release of a compressed gas in the sealed container; liquefied propellant techniques in which a low boiling liquid hydrocarbon (e.g., butane) is vaporized to exert a pressure and force the composition through the metered valve; and the like. Powders may be administered, for example, in such a manner that they are placed in a capsule that is then set in an inhalation or insufflation device. A needle is penetrated through the capsule to make pores at the top and the bottom of the capsule and air is sent to blow out the powder particles. Powder formulation can also be administered in a jet-spray of an inert gas or suspended in liquid organic fluids.

In one embodiment, the invention provides a nasally administrable pharmaceutical composition comprising at least one cholinesterase inhibitor dispersed in a nasal delivery system that improves the solubility of the cholinesterase inhibitor. The nasal delivery system that improves solubility can include one of the following or combinations thereof: (i) a glycol derivative (e.g., propylene glycol, polyethylene glycol, mixtures thereof); (ii) a system that improves its solubility.

In yet another embodiment, the invention provides a nasally administrable pharmaceutical composition comprising at least one cholinesterase inhibitor and a nasal delivery system, where the nasal delivery system comprises at least one pharmaceutically acceptable thickening agent, at least one humectant, at least one surfactant, and at least one solubilizing agent. The nasal delivery system can optionally further comprise buffers, pH adjusting agents, isotonicity agents, preservatives, antioxidants, bio-adhesives, and/or other pharmaceutically acceptable excipients. The cholinesterase inhibitor can optionally be dispersed in a nasal delivery system that improves its solubility.

In yet another embodiment, the invention provides a nasally administrable pharmaceutical composition comprising at least one cholinesterase inhibitor and a nasal delivery system, where the nasal delivery system comprises at least one buffer to maintain the pH of the cholinesterase inhibitor, at least one pharmaceutically acceptable thickening agent, at least one humectant, at least one surfactant, and at least one solubilizing agent. The nasal delivery system can optionally further comprise buffers, pH adjusting agents, isotonicity agents, preservatives, antioxidants, bio-adhesives, and/or other pharmaceutically acceptable excipients. The cholinesterase inhibitor can optionally be dispersed in a nasal delivery system that improves its solubility.

The nasally administrable pharmaceutical compositions of the invention preferably provide a peak plasma concentration of the cholinesterase inhibitor in less than one hour, preferably within about 5 minutes to about 30 minutes, more preferably within about 5 minutes to about 20 minutes, after administration to the patient.

The buffer has a pH that is selected to optimize the absorption of the cholinesterase inhibitor across the nasal mucosa. The particular pH of the buffer can vary depending upon the particular nasal delivery formulation as well as the specific cholinesterase inhibitor selected. Buffers that are suitable for use in the invention include acetate (e.g., sodium acetate), citrate (e.g., sodium citrate dihydrate), phthalate, borate, prolamine, trolamine, carbonate, phosphate (e.g., monopotassium phosphate, disodium phosphate), and mixtures of two or more thereof.

The pH of the compositions should be maintained from about 3.0 to about 10.0. Compositions having a pH of less than about 3.0 or greater than about 10.0 can increase the risk of irritating the nasal mucosa of the patient. Further, it is preferable that the pH of the compositions be maintained from about 3.0 to about 9.0. With respect to the non-aqueous nasal formulations, suitable forms of buffering agents can be selected such that when the formulation is delivered into the nasal cavity of a mammal, selected pH ranges are achieved therein upon contact with, e.g., a nasal mucosa.

The solubilizing agent for use in the compositions of the invention can be any known in the art, such as carboxylic acids and salts thereof. Exemplary carboxylic acid salts include acetate, gluconate, ascorbate, citrate, fumurate, lactate, tartrate, maleate, maleate, succinate, or mixtures of two or more thereof.

The viscosity of the compositions of the present invention can be maintained at a desired level using a pharmaceutically acceptable thickening agent. For example, the viscosity may be at least 1000 cps; from about 1000 to about 10,000 cps; from about 2000 cps to about 6500 cps; or from about 2500 cps to about 5000 cps. Thickening agents that can be used in accordance with the present invention include, for example, methyl cellulose, xanthan gum, carboxymethyl cellulose, hydroxypropyl cellulose, carbomer, polyvinyl alcohol, alginates, acacia, chitosans, and mixtures of two or more thereof. The concentration of the thickening agent will depend upon the agent selected and the viscosity desired. Such agents can also be used in a powder formulation.

The nasally administrable compositions can also include a humectant to reduce or prevent drying of the mucus membrane and to prevent irritation thereof. Suitable humectants that can be used include, for example, sorbitol, mineral oil, vegetable oil and glycerol; soothing agents; membrane conditioners; sweeteners; and mixtures of two or more thereof. The concentration of the humectant will vary depending upon the agent selected. In one embodiment, the humectant can be present in the nasal delivery system in a concentration ranging from about 0.01% to about 20% by weight of the composition.

In other embodiments, the nasal delivery system can further comprise surfactants which enhance the absorption of the cholinesterase inhibitor. Suitable surfactants include non-ionic, anionic and cationic surfactants. Exemplary surfactants include oleic acid, polyoxyethylene derivatives of fatty acids, partial esters of sorbitol anhydride, such as for example, Tweens (e.g., Tween 80, Tween 40, Tween 20), Spans (e.g., Span 40, Span 80, Span 20), polyoxyl 40 stearate, polyoxy ethylene 50 stearate, fusieates, bile salts, octoxynol, and mixtures of two or more thereof. Exemplary anionic surfactants include salts of long chain hydrocarbons (e.g., C₆₋₃₀ or C₁₀₋₂₀) having one or more of the following functional groups: carboxylates; sulfonates; and sulfates. Salts of long chain hydrocarbons having sulfate functional groups are preferred, such as sodium cetostearyl sulfate, sodium dodecyl sulfate and sodium tetradecyl sulfate. One particularly preferred anionic surfactant is sodium lauryl sulfate (i.e., sodium dodecyl sulfate). The surfactants can be present in an amount from about 0.001% to about 50% by weight, or from about 0.001% to about 20% by weight.

The pharmaceutical compositions of the invention may further comprise an isotonicity agent, such as sodium chloride, dextrose, boric acid, sodium tartrate or other inorganic or organic solutes.

The nasal pharmaceutical compositions of the invention can optionally be used in combination with a pH adjusting agent. Exemplary pH adjusting agents include sulfuric acid, sodium hydroxide, hydrochloric acid, and the like.

To extend shelf life, preservatives can be added to the nasally administrable compositions. Suitable preservatives that can be used include benzyl alcohol, parabens, thimerosal, chlorobutanol, benzalkonium chloride, or mixtures of two or more thereof. Preferably benzalkonium chloride is used. Typically, the preservative will be present in a concentration of up to about 2% by weight. The exact concentration of the preservative, however, will vary depending upon the intended use and can be easily ascertained by one skilled in the art.

Other ingredients which extend shelf life can be added such as for example, antioxidants. Some examples of antioxidants include sodium metabisulfite, potassium metabisulfite, ascorbyl palmitate and the like. Typically, the antioxidant will be present in the compositions in a concentration of from about 0.001% up to about 5% by weight of the total composition.

Other optional ingredients can also be incorporated into the nasal delivery system provided that they do not interfere with the action of the cholinesterase inhibitor or significantly decrease the absorption of the cholinesterase inhibitor across the nasal mucosa.

The nasal delivery systems can be made following the processes described in, for example, U.S. Pat. Nos. 6,451,848, 6,436,950, and 5,874,450, and WO 00/00199, the disclosures of which are incorporated by reference herein in their entirety.

In another embodiment, the invention provides methods for treating and/or preventing migraines by nasally administering to a patient the nasally administrable pharmaceutical composition comprising at least one cholinesterase inhibitor of the invention. The migraines can be classic migraines, common migraines, complicated migraines, and/or cluster headaches. In other embodiments, the migraines can be menstrual migraines, premenstrual migraines, ophthalmic migraines, and/or ophthalmoplegic migraines. In other embodiments, the migraines can be fulgurating migraines, Harris' migraines, and/or hemiplegic migraines. In still other embodiments, the migraines can be abdominal migraines.

Each of the patents, patent applications, and publications cited herein are incorporated by reference herein in their entirety.

It will be apparent to one skilled in the art that various modifications can be made to the invention without departing from the spirit or scope of the appended claims.

1. (canceled)

2. (canceled)

3. (canceled)

4. (canceled)

5. (canceled)

6. (canceled)

7. (canceled)

8. (canceled)

9. A method for treating incontinence in a patient in need thereof comprising administering a therapeutically effective amount of at least one cholinesterase inhibitor.

10. (canceled)

11. (canceled)

12. (canceled)

13. (canceled)

14. (canceled)

15. The method of claim 9, wherein the cholinesterase inhibitor is donepezil, phenserine, tolserine, phenethylnorcymserine, ganstigmine, epastigmine, tacrine, physostigmine, pyridostigmine, neostigmine, rivastigmine, galantamine, citicoline, velnacrine, huperzine, metrifonate, heptastigmine, edrophonium, 3-(1-(phenylmethyl)-4-piperidinyl)-1-(2,3,4,5-tetrahydro-1H-1-benzazepin-8-yl)-1-propanone, T-82 or upreazine.

16. The method of claim 9, wherein the cholinesterase inhibitor is a compound of formula I, a stereoisomer thereof, and/or a pharmaceutically acceptable salt thereof: 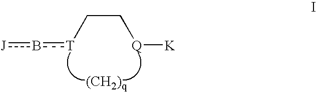
wherein J is
a substituted or unsubstituted phenyl
B is —(CHR²²)ᵣ—, —CO—(CHR²²)ᵣ—, —NR4-(CHR²²)ᵣ—, —CO—NR⁵—(CHR²²)ᵣ—, —CH═CH—(CHR²²)ᵣ—, —OCOO—(CHR²²)ᵣ—, —OOC—NH—(CHR²²)ᵣ—, —NH—CO—(CHR²²)ᵣ—, —CH₂—CO—NH—(CHR²²)ᵣ—, —(CH₂)₂—NH—(CHR²²)ᵣ—, —CH(OH)—(CHR²²)ᵣ—, ═(CH—CH═CH)_{b}—, ═CH—(CH₂)_{c}—, ═(CH—CH)_{d}═, —CO—CH═CH—CH₂—, —CO—CH₂—CH(OH)—CH₂—, —CH(CH₃)—CO—NH—CH₂—, —CH═CH═CO—NH—(CH₂)₂—, —NH—, —O—, —S—, a dialkylaminoalkyl-carbonyl or a lower alkoxycarbonyl;
wherein R⁴ is hydrogen, lower alkyl, acyl, lower alkylsulfonyl, phenyl, substituted phenyl, benzyl, or substituted benzyl; R⁵ is hydrogen, lower alkyl or phenyl; r is zero or an integer of about 1 to about 10; R²² is hydrogen or methyl so that one alkylene group can have no methyl branch or one or more methyl branches; b is an integer of about 1 to about 3; c is zero or an integer of about 1 to about 9; d is zero or an integer of about 1 to about 5;
T is nitrogen or carbon
Q is nitrogen, carbon or 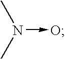
q is an integer of about 1 to about 3;
K is hydrogen, phenyl, substituted phenyl, arylalkyl in which the phenyl can have a substituent, cinnamyl, a lower alkyl, pyridylmethyl, cycloalkylalkyl, adamantanemethyl, furylmenthyl, cycloalkyl, lower alkoxycarbonyl or an acyl; and [symbol]
is a single bond or a double bond.

17. The method of claim 9, wherein the cholinesterase inhibitor is a compound of formula II, a stereoisomer thereof, and/or a pharmaceutically acceptable salt thereof: 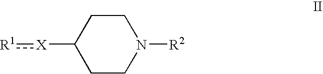
wherein R¹ is a substituted or unsubstituted phenyl group; X is —(CH₂)ₙ—, —C(O)—(CH₂)ₙ—, —N(R⁴)—(CH₂)ₙ—, —C(O)—N(R⁵)—(CH₂)ₙ—, —CH═CH—(CH₂)ₙ—, —O—C(O)—O—(CH₂)ₙ—, —O—C(O)—NH—(CH₂)ₙ—, —CH═CH—CH═CO—, —NH—C(O)—(CH₂)ₙ—, —CH₂—C(O)—NH—(CH₂)ₙ—, —(CH₂)₂—C(O)—NH—(CH₂)ₙ—, —CH(OH)—(CH₂)ₙ—, —C(O)—CH═CH—CH₂—, —C(O)—CH₂—CH(OH)—CH₂—, —CH(CH₃)—C(O)—NH—CH₂—, —CH═CH—C(O)—NH—(CH₂)₂—, a dialkylaminoalkylcarbonyl group, a lower alkoxycarbonyl group;
where n is an integer of 0 to 6; R⁴ is a hydrogen atom, a lower alkyl group, an acyl group, a lower alkylsulfonyl group, a substituted or unsubstituted phenyl group, or a substituted or unsubstituted benzyl group; and R⁵ is a hydrogen atom a lower alkyl group or a phenyl group;
R² is a substituted or unsubstituted phenyl group; a substituted or unsubstituted arylalkyl group; a cinnamyl group; a lower alkyl group; a pyridylmethyl group; a cycloalkylalkyl group; an adamantanemethyl group; or a furoylmethyl group; and [symbol]
is a single bond or a double bond.

18. The method of claim 9, wherein the cholinesterase inhibitor is a compound of formula III, a stereoisomer thereof, and/or a pharmaceutically acceptable salt thereof: 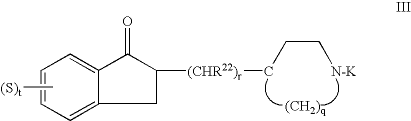
wherein r is an integer of about 1 to about 10; each R²² is independently hydrogen or methyl; K is a phenalkyl or a phenalkyl having a substituent on the phenyl ring; each S is independently a hydrogen, a lower alkyl group having 1 to 6 carbon atoms or a lower alkoxy group having 1 to 6 carbon atoms; t is an integer of 1 to 4; q is an integer of about 1 to about 3; with the proviso that (S)ₜ can be a methylenedioxy group or an ethylenedioxy group joined to two adjacent carbon atoms of the phenyl ring.

19. The method of claim 9, wherein the cholinesterase inhibitor is 1-benzyl-4-((5,6-dimethoxy-1-indanon)-2-yl)methylpiperidine, 1-benzyl-4-((5,6-dimethoxy-1-indanon)-2-ylidenyl)methylpiperidine; 1-benzyl-4-((5-methoxy-1-indanon)-2-yl)methylpiperidine; 1-benzyl-4-((5,6-diethoxy-1-indanon)-2-yl)methylpiperidine; 1-benzyl-4-((5,6-methnylenedioxy-1-indanon)-2-yl)methylpiperidine; 1-(m-nitrobenzyl)-4-((5,6-dimethoxy-1-indanon)-2-yl)methylpiperidine; 1-cyclohexylmethyl-4-((5,6-dimethoxy-1-indanon)-2-yl)methylpiperidine; 1-(m-fluorobenzyl)-4-((5,6-dimethoxy-1-indanon)-2-yl)methylpiperidine; 1-benzyl-4-((5,6-dimethoxy-1-indanon)-2-yl)propylpiperidine; 1-benzyl-4-((5-isopropoxy-6-methoxy-1-indanon)-2-yl)methylpiperidine; 1-benzyl-4-((5,6-dimethoxy-1-oxoindanon)-2-yl)propenylpiperidine; or a stereoisomer and/or a pharmaceutically acceptable salt thereof.

20. The method of claim 9, wherein the cholinesterase inhibitor is a compound of formula IV or a pharmaceutically acceptable salt thereof:

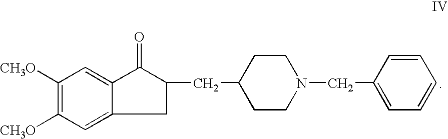

21. The method of claim 9, wherein the cholinesterase inhibitor

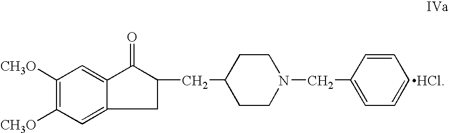

22. The method of claim 9, wherein the cholinesterase inhibitor is a compound of formula VI or a pharmaceutically acceptable salt thereof:

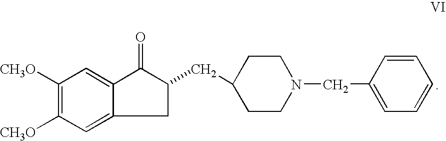

23. The method of claim 9, wherein the cholinesterase inhibitor is a compound of formula VII or a pharmaceutically acceptable salt thereof: [chemical structure]
